(19) United States
(12) Patent Application Publication
(10) Pub. No.: US 2023/0090551 A1
(43) Pub. Date: Mar. 23, 2023

(54) COMPOSITION, KIT AND METHOD FOR DETECTING SARS-COV-2 AND USE THEREOF

(71) Applicant: SANSURE BIOTECH INC., Changsha (CN)

(72) Inventors: Lizhong DAI, Changsha (CN); Zhongping DENG, Changsha (CN); Deyong TAN, Changsha (CN); Jia LIU, Changsha (CN); Bozhi JI, Changsha (CN); Jin YAN, Changsha (CN); Xinwu GUO, Changsha (CN); Xiaomei REN, Changsha (CN); Kang WU, Changsha (CN); Xing CHENG, Changsha (CN)

(21) Appl. No.: 17/896,580

(22) Filed: Aug. 26, 2022

Related U.S. Application Data
(63) Continuation of application No. PCT/CN2020/121026, filed on Oct. 15, 2020.

(30) Foreign Application Priority Data
Jun. 3, 2020 (CN) 202010496832.2

(51) Int. Cl. C12Q 1/70

(52) U.S. Cl. CPC C12Q 1/701; C12Q 1/6851

(57) ABSTRACT

A composition for detecting SARS-CoV-2 is provided; moreover, a kit including the composition, the use of the kit, and a method for detecting SARS-CoV-2 are also provided.

### CROSS-REFERENCE TO RELATED APPLICATIONS

This application is a continuation of International Application No. PCT/CN2020/121026, filed on Oct. 15, 2020, which claims priority to Chinese Patent Application No. 202010496832.2, filed on Jun. 3, 2020. The disclosures of the aforementioned applications are hereby incorporated by reference in their entireties.

### REFERENCE TO AN ELECTRONIC SEQUENCE LASTING

The contents of the electronic sequence listing (CU698SequenceListing.xml; Size: 53; 428 bytes; and Date of Creation: Aug. 26, 2022) is herein incorporated by reference in its entirety.

### TECHNICAL FIELD

The present invention relates to the field of molecular biological detection, and in particular, to detection of SARS-CoV-2.

### BACKGROUND

Novel coronavirus (2019-nCoV, SARS-CoV-2) is named SARS-CoV-2 by the International Committee on Taxonomy of Viruses on February 11. Coronavirus is a large family of viruses, six viruses of which are previously known to infect humans, such as causing influenza, Middle East Respiratory Syndrome (MERS) and Severe Acute Respiratory Syndrome (SARS). SARS-CoV-2 is a new strain of coronavirus that has never been found in humans before. The virus is taxonomically a β-type coronavirus in the genus Coronavims of the family Coronaviridae. The virus has envelope and spike cytology features, and its genome is a linear single-strand positive-stranded RNA ((+)ssRNA). Since the discovery of SARS-CoV-2 at the end of 2019, in just four months, it breaks out almost all over the world, causing more than 1.4 million infections and more than 70,000 deaths in the world, which has attracted great attention from the international community.

In the current “Novel Coronavirus Pneumonia Diagnosis and Treatment Plan (Trial Version 7)”, the detection of SARS-CoV-2 nucleic acid is still the only basis for confirmed diagnosis. There are two ways to clarify nucleic acid detection in the diagnosis and treatment plan: (1) real-time quantitative PCR (qPCR) detection of nucleic acid positive; and (2) viral gene sequencing, the sequencing result being highly homologous to known SARS-CoV-2.

Although the viral gene sequencing method is more accurate, it is time-consuming and expensive, and it is difficult to apply clinically on a large scale.

Due to the above-mentioned limitations of gene sequencing, most of patients with high suspicion to be excluded are tested by qPCR clinically. Compared with the gene sequencing, qPCR has the advantages of high speed, batch size, and low price, etc.

It is reported that since the large-scale use of qPCR detection reagents for clinical diagnosis, the initial nucleic acid positive detection rate is only 30%-50%. In addition, some medical institutions have reported that the results of a few cases have changed from negative to positive after five or more detections at different times. There are many reports on the Internet at home and abroad that primers and probes provided by the WHO have specificity problems, detection reagents provided by the CDC of the United States have the problem of poor detection effect, and after recovery and discharge from hospital, it is again found that the SARS-CoV-2 nucleic acid detection turns positive. The occurrence of these situations has brought great troubles to clinical diagnosis and disease control.

Therefore, there is a need in the art for a more sensitive and accurate qPCR-related product for detecting SARS-CoV-2.

### SUMMARY

To solve the problems above, the inventors have conducted numerous analytical and experimental studies, and found two specific areas on an ORF1ab gene (Genbank ID: NC_045512.2 (266 . . . 21555)) in the genome of SARS-CoV-2, to design amplification primers and probes for qPCR detection, the nucleic acid positive detection rate can be effectively improved, and the present invention is completed accordingly.

The two specific areas mentioned above are respectively:

(SEQ ID NO: 1)
ACAATTCACCTAATTTAGCATGGCCTCTTATTGTAACAGCT
TTAAGGGCCAATTCTGCTGTCAAATTACAGAATAATGAGCT
TAGTCCTGTTGCACTACGACAGATGTCTTGTGCTGCCGGTA
CTACACAAACTGCTTGCACTGATGACAATGCGTTAGCTTAC
TACAAC;
and
(SEQ ID NO: 2)
ACTAGAAGGTTCTGTTGCTTATGAAAGTTTACGCCCTGACA
CACGTTATGTGCTCATGGATGGCTCTATTATTCAATTTCCT
AACACCTACCTTGAAGGTTCTGTTAGAGTGGTAACAACTTT
TGATTCTGAGTACTGTAGGCACGGCACTTGTGAAAGATCAG
AAGCTG.

Therefore, in a first aspect, the present invention provides a composition for detecting SARS-CoV-2, including:

a first primer pair, used for specifically amplifying an N gene of SARS-CoV-2;

a first probe, used for detecting an amplification product of the first primer pair;

a second primer pair, used for specifically amplifying an area as shown in SEQ ID NO: 1 in an ORF1ab gene;

a second probe, used for detecting an amplification product of the second primer pair;

a third primer pair, used for specifically amplifying an area as shown in SEQ ID NO: 2 in the ORF1ab gene; and

a third probe, used for detecting an amplification product of the third primer pair.

The primers and probes used in the present invention are all applied to qPCR.

By simultaneously detecting two specific areas of the ORF1ab gene and the N gene, the composition of the present invention improves the nucleic acid positive detection rate of SARS-CoV-2, makes the detection result more accurate, and thus has positive significance for epidemic prevention and control and the treatment of confirmed patients.

In preferred embodiments, the first primer pair of the present invention specifically amplifies the following areas of the N gene (Genbank ID: NC_045512.2 (28274 . . . 29533)):

(SEQ ID NO: 3)
ATGGTATTTCTACTACCTAGGAACTGGGCCAGAAGCTGGACT
TCCCTATGGTGCTAACAAAGACGGCATCATATGGGTTGCAAC
TGAGGGAGCCTTGAATACACCAAAAGATCACATTGGCACCCG
CAATCCTGCTAACAATGCTGCAATCG.

In a specific embodiment, the first primer pair and the first probe of the present invention are:

an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
an N gene probe 1:
(SEQ ID NO: 7)
5′-GCCAGAAGCTGGACTTCCCTATGGTGC-3′.

In a specific embodiment, the first primer pair and the first probe of the present invention are:

an N gene forward primer 2:
(SEQ ID NO: 43)
5′-ACTACCTAGGAACTGGGCCAG-3′;
an N gene reverse primer 2:
(SEQ ID NO: 44)
5′-AGCAGGATTGCGGGTGCCAAT-3′;
and
an N gene probe 2:
(SEQ ID NO: 45)
5′-ACTTCCCTATGGTGCTAACAAAGACGGCAT-3′.

By using the primers and probes of the present invention for detecting N gene, Ct values of the N gene are significantly moved forward, it means that the lower limit of detection of the N gene is decreased, and the N gene at a lower concentration can be detected, which also helps to detect SARS-CoV-2 of lower concentration, making the detection more sensitive and accurate.

In a specific embodiment, the second primer pair and the second probe of the present invention are:

an ORF1ab forward primer 1-1:
(SEQ ID NO: 8)
5′-ATTCACCTAATTTAGCATGGCCTC-3′;
an ORF1ab reverse primer 1-1:
(SEQ ID NO: 9)
5′-ACATCTGTCGTAGTGCAACAGGAC-3′;
and
an ORF1ab probe 1-1:
(SEQ ID NO: 10)
5′-TTGTAACAGCTTTAAGGGCCAATTCTGCTGT-3′.

In a specific embodiment, the second primer pair and the second probe of the present invention are:

an ORF1ab forward primer 1-2:
(SEQ ID NO: 1)
5′-ATGGCCTCTTATTGTAACAGCT-3′;
an ORF1ab reverse primer 1-2:
(SEQ ID NO: 12)
5′-AGCAGTTTGTGTAGTACCGGCAGC-3′;
and
an ORF1ab probe 1-2:
(SEQ ID NO: 13)
5′-CCAATTCTGCTGTCAAATTACAGAATA-3′.

In a specific embodiment, the third primer pair and the third probe of the present invention are:

an ORF1ab forward primer 2-1:
(SEQ ID NO: 14)
5′-GAAGGTTCTGTTGCTTATGAAAGTT-3′;
an ORF1ab reverse primer 2-1:
(SEQ ID NO: 15)
5′-TACCACTCTAACAGAACCTTC-3′;
and
an ORF1ab probe 2-1:
(SEQ ID NO: 16)
5′-GCCCTGACACACGTTATGTGCTCATGGA-3′.

In a specific embodiment, the third primer pair and the third probe of the present invention are:

an ORF1ab forward primer 2-2:
(SEQ ID NO: 17)
5′-ATGAAAGTTTACGCCCTGAC-3′;
an ORF1ab reverse primer 2-2:
(SEQ ID NO: 18)
5′-CCGTGCCTACAGTACTCAGAATC-3′;
and
an ORF1ab probe 2-2:
(SEQ ID NO: 19)
5′-ATGTGCTCATGGATGGCTCTATTATTCAATT-3′.

The respective specific primer pair and probe sequences respectively of the first primer pair and the first probe, the second primer pair and the second probe, and the third primer pair and the third probe can be combined with each other, as long as satisfying that each composition includes the first primer pair and the first probe, the second primer pair and the second probe, and the third primer pair and the third probe.

In an exemplary embodiment, the present invention provides a first composition, including:

a first primer pair:
an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
a first probe:
an N gene probe 1:
(SEQ ID NO: 7)
5′-GCCAGAAGCTGGACTTCCCTATGGTGC-3′;
a second primer pair:
an ORF1ab forward primer 1-1:
(SEQ ID NO: 8)
5′-ATTCACCTAATTTAGCATGGCCTC-3′;
an ORF1ab reverse primer 1-1:
(SEQ ID NO: 9)
5′-ACATCTGTCGTAGTGCAACAGGAC-3′;
and
a second probe:
an ORF1ab probe 1-1:
(SEQ ID NO: 10);
5′-TTGTAACAGCTTTAAGGGCCAATTCTGCTGT-3′
and
a third primer pair:
an ORF1ab forward primer 2-1:
(SEQ ID NO: 14)
5′-GAAGGTTCTGTTGCTTATGAAAGTT-3′;
an ORF1ab reverse primer 2-1:
(SEQ ID NO: 15)
5′-TACCACTCTAACAGAACCTTC-3′;
and
a third probe:
an ORF1ab probe 2-1:
(SEQ ID NO: 16)
5′-GCCCTGACACACGTTATGTGCTCATGGA-3′.

In another exemplary embodiment, the present invention provides a second composition, including:

a first primer pair:
an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
a first probe:
an N gene probe 1:
(SEQ ID NO: 7)
5′-GCCAGAAGCTGGACTTCCCTATGGTGC-3′;
a second primer pair:
an ORF1ab forward primer 1-2:
(SEQ ID NO: 11)
5′-ATGGCCTCTTATTGTAACAGCT-3′;
an ORF1ab reverse primer 1-2:
(SEQ ID NO: 12)
5′-AGCAGTTTGTGTAGTACCGGCAGC-3′;
and
a second probe:
an ORF1ab probe 1-2:
(SEQ ID NO: 13)
5′-CCAATTCTGCTGTCAAATTACAGAATA-3′;
and
a third primer pair:
an ORF1ab forward primer 2-2:
(SEQ ID NO: 17)
5′-ATGAAAGTTTACGCCCTGAC-3′;
an ORF1ab reverse primer 2-2:
(SEQ ID NO: 18)
5′-CCGTGCCTACAGTACTCAGAATC-3′;
and
a third probe:
an ORF1ab probe 2-2:
(SEQ ID NO: 19)
5′-ATGTGCTCATGGATGGCTCTATTATTCAATT-3′.

In yet another exemplary embodiment, the present invention provides a third composition, including:

a first primer pair:
an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
a first probe:
an N gene probe 1:
(SEQ ID NO: 7)
5′-GCCAGAAGCTGGACTTCCCTATGGTGC-3′;
a second primer pair:
an ORF1ab forward primer 1-1:
(SEQ ID NO: 8)
5′-ATTCACCTAATTTAGCATGGCCTC-3′;
an ORF1ab reverse primer 1-1:
(SEQ ID NO: 9)
5′-ACATCTGTCGTAGTGCAACAGGAC-3′;
and
a second probe:
an ORF1ab probe 1-1:
(SEQ ID NO: 10)
5′-TTGTAACAGCTTTAAGGGCCAATTCTGCTGT-3′;
and
a third primer pair:
an ORF1ab forward primer 2-2:
(SEQ ID NO: 17)
5′-ATGAAAGTTTACGCCCTGAC-3′;
an ORF1ab reverse primer 2-2:
(SEQ ID NO: 18)
5′-CCGTGCCTACAGTACTCAGAATC-3′;
and
a third probe:
an ORF1ab probe 2-2:
(SEQ ID NO: 19)
5′-ATGTGCTCATGGATGGCTCTATTATTCAATT-3′

In still another exemplary embodiment, the present invention provides a fourth composition, including:

a first primer pair:
an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
a first probe:
an N gene probe 1:
(SEQ ID NO: 7)
5′-GCCAGAAGCTGGACTTCCCTATGGTGC-3′;
a second primer pair:
an ORF1ab forward primer 1-2:
(SEQ ID NO: 11)
5′-ATGGCCTCTTATTGTAACAGCT-3′;
an ORF1ab reverse primer 1-2:
(SEQ ID NO: 12)
5′-AGCAGTTTGTGTAGTACCGGCAGC-3′;
and
a second probe:
an ORF1ab probe 1-2:
(SEQ ID NO: 13)
5′-CCAATTCTGCTGTCAAATTACAGAATA-3′;
and
a third primer pair:
an ORF1ab forward primer 2-1:
(SEQ ID NO: 14)
5′-GAAGGTTCTGTTGCTTATGAAAGTT-3′;
an ORF1ab reverse primer 2-1:
(SEQ ID NO: 15)
5′-TACCACTCTAACAGAACCTTC-3′;
and
a third probe:
an ORF1ab probe 2-1:
(SEQ ID NO: 16)
5′-GCCCTGACACACGTTATGTGCTCATGGA-3′.

By using the composition as shown above, the present invention makes the positive rate of nucleic acid detection of SARS-CoV-2 higher, and moreover can detect SARS-CoV-2 with higher sensitivity, which has a positive significance for the epidemic prevention and control and the treatment of the confirmed patients.

In a specific embodiment, the composition of the present invention may further include primer pairs that can further perform nested PCR.

Furthermore, in the composition, the fluorescent reporter groups of the second probe and the third probe may be the same or different.

Furthermore, in the composition, the first probe, the second probe and the third probe carry fluorescent reporter groups that do not interfere with each other.

Herein, “not interfere with each other” means that the fluorescent reporter groups used by the probes in the composition are different, and may not affect each other's detection, that is, different channels can be used for detection. For example, FAM, HEX, ROX and CY5 can be used. These groups do not have close absorbance values and can select different channels, and thus may not interfere with each other.

In the present invention, the fluorescent reporter group can be selected from a group consisting of FAM, HEX, ROX, VIC, CY5, 5-TAMRA, TET, CY3, and JOE, and is not limited thereto.

In a specific embodiment, the fluorescent reporter group of the first probe is ROX, and the fluorescent reporter groups of the second and third probes are FAM.

Furthermore, the 3′ end of the probe also has a quenching group such as BHQ1 or BHQ2.

Furthermore, the composition includes an internal standard upstream primer, an internal standard downstream primer and an internal standard probe for monitoring.

The internal standard gene is an RNaseP gene (Genbank ID: U77665.1). Preferably, the internal standard upstream primer and the internal standard downstream primer specifically amplify the following sequence:

(SEQ ID NO: 4)
GAATTCGGCACGAGGTGGGACTTCAGCATGGCGGTGTTTGCAGATTTGGACC
TGCGAGCGGGTTCTGACCTGAAGGCTCTGCGCGGACTTGTGGAGACAGCCGC
TCACCTTGGCTATTCAGTTGTTGCTATCAATCATATCGTTGACTTTAAGGAA
AAGAAACAGGAAATTGAAAAACCAGTAGCTGTTTCTGAACTCTTCACAACTT
TGCCAATTGTACAGGGAAAATCAAGACCAATTAAAATTTTAACTAGATTAAC
AATTATTGTC.

In a specific embodiment, the composition includes:

an internal standard primer pair, used for specifically amplifying an area of an RNaseP gene as shown in SEQ ID NO: 4; and

an internal standard probe, used for detecting an amplification product of the internal standard primer pair.

In a specific embodiment, the composition further includes:

an internal standard upstream primer:
(SEQ ID NO: 20)
5′-GAGGTGGGACTTCAGCATGGC-3′;
an internal standard downstream primer:
(SEQ ID NO: 21)
5′-TGTTTCTTTTCCTTAAAGTCAAC-3′;
and
an internal standard probe:
(SEQ ID NO: 22)
5′-AGATTTGGACCTGCGAGCGGGTTCTGA-3′.

Furthermore, the first, second and third probes and the internal standard probe carry fluorescent reporter groups that do not interfere with each other.

Furthermore, the fluorescent reporter group of the internal standard probe is HEX.

Furthermore, the dosage of the primer in the composition is 25-200 nM, and the dosage of the probe in the composition is 10-80 nM.

In some embodiments, the compositions of the present invention are present in the same package.

In other embodiments, the first primer pair and the first probe, the second primer pair and the second probe, and the third primer pair and the third probe of the present invention are present in separate packages, respectively.

In yet other embodiments, the first primer pair and the first probe of the present invention are present in one package, and the second primer pair and the second probe, and the third primer pair and the third probe are present in another package.

In still other embodiments, the first primer pair and the first probe, the second primer pair and the second probe, and the third primer pair and the third probe, and the internal standard primer pair and the internal standard probe of the present invention are present in separate packages, respectively.

In a second aspect, the present invention provides use of the composition of the present invention in preparation of a kit for detecting SARS-CoV-2.

In a third aspect, the present invention provides a kit for detecting SARS-CoV-2, including the composition of the present invention as described above.

Furthermore, the kit further includes at least one of dNTP, PCR buffer, and Mg²⁺.

Furthermore, the kit further includes at least one of a nucleic acid release reagent, a nucleic acid extraction reagent, reverse transcriptase, and DNA polymerase.

Furthermore, the kit further includes at least one of the nucleic acid release reagent, the nucleic acid extraction reagent, dNTP, the reverse transcriptase, the DNA polymerase, the PCR buffer, and Mg²⁺.

Furthermore, the concentration of the reverse transcriptase is 5-15 U/μL. For example, the reverse transcriptase can be murine leukemia reverse transcriptase (MMLV) or Tth enzyme. The concentration of the DNA polymerase is 3-15 U/μL. For example, the DNA polymerase can be Taq enzyme.

In a specific embodiment, the kit of the present invention includes an inverse/reverse transcriptase, a Taq enzyme, Mg²⁺, Mn²⁺, Rnasin, dNTP, a primer, a probe, and a PCR buffer.

The common PCR buffer is composed of buffer systems such as Tris-HCl, MgCl₂, KCl, and TritonX-100. In general, the total volume of a single PCR reaction tube is 20-200 μl.

In a specific embodiment, the kit of the present invention is compatible with a digital PCR amplification system, that is, it can be directly used for amplification on a digital PCR instrument.

In a fourth aspect, provided is a method for detecting SARS-CoV-2, including the following steps:

1) extracting or releasing nucleic acids from a to-be-tested sample;

2) using the composition of the present invention or the kit of the present invention to perform fluorescent quantitative PCR on the nucleic acids obtained in step 1); and

3) obtaining and analyzing a result.

In the present invention, a sample for detection can be a throat swab, sputum, bronchoalveolar lavage fluid, blood, etc., but is not limited thereto.

Furthermore, the reaction conditions of the fluorescent quantitative PCR are:

reverse transcription in which the temperature is 50° C. to 60° C., and the time is 15-35 minutes, 1 cycle; cDNA pre-denaturation in which the temperature is 95° C., and the time is 1-10 minutes, 1 cycle; denaturation in which the temperature is 95° C., and the time is 10-20 seconds; and annealing in which the temperature is 60° C., and the time is 20-60 seconds, 40-50 cycles.

In a specific embodiment, provided is a method for detecting SARS-CoV-2 for non-diagnostic purposes, including the following steps:

1) extracting or releasing nucleic acids from a to-be-tested sample;

2) using the composition of the present invention or the kit of the present invention to perform fluorescent quantitative PCR on the nucleic acids obtained in step 1); and

3) obtaining and analyzing a result.

Furthermore, the reaction conditions of the fluorescent quantitative PCR are:

reverse transcription in which the temperature is 50° C. to 60° C., and the time is 15-35 minutes, 1 cycle; cDNA pre-denaturation in which the temperature is 95° C., and the time is 1-10 minutes, 1 cycle; denaturation in which the temperature is 95° C., and the time is 10-20 seconds; and annealing in which the temperature is 60° C., and the time is 20-60 seconds, 40-50 cycles.

Herein, the term “non-diagnostic purposes” refers to information that is not intended to obtain whether an individual is infected with SARS-CoV-2 and has pneumonia. For example, the method can detect the presence or absence of SARS-CoV-2 in test cultures in experiments for research purposes.

### BRIEF DESCRIPTION OF THE DRAWINGS

FIG. 1 is a detection result diagram of a first composition of the composition of the present invention for detecting SARS-CoV-2;

FIG. 2 is a comparison diagram of the detection effects of the first composition of the composition of the present invention and a single-primer probe composition of ORF1ab;

FIG. 3 is a comparison diagram of the detection effects of the first composition of the composition of the present invention and another single-primer probe composition of ORF1ab;

FIG. 4 is a comparison diagram of the detection effects of N gene primer probes of the composition of the present invention and primer probes of the CDC of China and the CDC of the United States;

FIG. 5 is a comparison diagram of the detection effects of N gene primer probes of the composition of the present invention and primer probes of the University of Hong Kong;

FIG. 6 is a comparison diagram of the detection effects of the composition of the present invention of an ORF1ab gene and a comparative example composition (a fifth composition);

FIG. 7 is a comparison diagram of the detection effects of the composition of the present invention of the ORF1ab gene and another comparative example composition (a sixth composition);

FIG. 8 is a detection result diagram of a FAM channel of the composition of the present invention (a first composition, a third composition, or a fourth composition) for detecting SARS-CoV-2;

FIG. 9 is a detection result diagram of an ROX channel of a seventh composition of the present invention for detecting SARS-CoV-2;

FIG. 10 is a detection result diagram of an ROX channel of an eighth composition of the present invention for detecting SARS-CoV-2;

FIG. 11 is a detection result diagram of primer and probe systems designed for three specific areas of the ORF1ab gene; and

FIG. 12 is a detection result diagram of primer and probe systems designed for two specific areas of the N gene.

### DETAILED DESCRIPTION

In the present invention, the expressions “first”, “second”, “third” and “fourth” etc. are only used for descriptive purposes, to distinguish the defined substances, and not to define the order or priority in any way.

For a target area provided by the present invention (e.g., an area as shown in SEQ ID NO: 1, an area as shown in SEQ ID NO: 2, an area as shown in SEQ ID NO: 3, and/or an area as shown in SEQ ID NO: 4), specific amplification primers and corresponding detection probes can be designed. For example, it can be done in the following manner.

For the fluorescent quantitative PCR primers, the following principles are usually followed: 1. the primers should be designed in an area and have specificity; 2. the length of the amplification product is 80-150 bp, and the longest one should not exceed 300 bp; 3. the product cannot form a secondary structure (the free energy is less than 58.61 KJ/mol); 4. The primer length is generally between 17 bases and 25 bases, the upstream primer and the downstream primer should not differ too much; 5. the primer itself cannot have four consecutive bases of complementarity to avoid the formation of a hairpin structure; 6. the primers cannot have four consecutive bases of complementarity to avoid the formation of primer dimers; 7. the G+C content of the primers is between 40% and 60%, and 45% to 55% is the best, and the bases in the primers are randomly distributed, and evenly distributed to the greatest extent; and 8. the Tm value of the primers is between 58 degrees and 62 degrees.

According to the principles above, for the target area provided by the present invention, for example, the primers, including, but not limited to, the primers as shown in SEQ ID NO: 5 and SEQ ID NO:6, SEQ ID NO:8 and SEQ ID NO:9, SEQ ID NO:11 and SEQ ID NO:12, SEQ ID NO:14 and SEQ ID NO:15, SEQ ID NO:17 and SEQ ID NO:18, SEQ ID NO:20 and SEQ ID NO:21, SEQ ID NO:43 and SEQ ID NO:44 can be designed.

For the fluorescent quantitative PCR probes, the following principles are usually followed: 1. the probes are designed, and then the primers are designed to be close to the probes as far as possible; 2. the Tm value of the probes should be between 68 and 70, and in the case of visual probes, GC-rich areas are examined carefully; 3. a chain with more C than G is selected as the probe, and the content of G more than C may reduce the reaction efficiency; in this case, another paired chain should be selected as the probe; 4. the probe should be as short as possible, generally not more than 30 bp; and 5. the DNA folding and secondary structure of the probe is detected.

According to the principles above, for the target area provided by the present invention, for example, the primers, including, but not limited to, the primers as shown in SEQ ID NOs: 7, 10, 13, 16, 19, 22, and 45 can be designed.

Common primer and probe design software, such as Primer Express, Primer Premier, Oligo, and Omiga can be used, to assist in design. According to the specific amplification area provided by the present invention, those skilled in the art can use the software to assist in the design of probes and primers under the guidance of the above-mentioned principles.

Hereinafter, the present invention is described in detail with reference to specific embodiments and examples, and the advantages and various effects of the present invention may be more clearly presented therefrom. It should be understood by those skilled in the art that these specific embodiments and examples are intended to illustrate the present invention, instead of limiting the present invention.

### Example 1. Primers and Probes Used in the Present Invention

The primers and probes used in the present invention are as shown below:

a first composition:

a first primer pair: an N gene forward primer 1 (SEQ ID NO: 5) and an N gene reverse primer 1 (SEQ ID NO: 6);

a first probe: an N gene probe 1 (SEQ ID NO: 7);

a second primer pair: an ORF1ab forward primer 1-1 (SEQ ID NO: 8) and an ORF1ab reverse primer 1-1 (SEQ ID NO: 9);

a second probe: an ORF1ab probe 1-1 (SEQ ID NO: 10); and

a third primer pair: an ORF1ab forward primer 2-1 (SEQ ID NO: 14) and an ORF1ab reverse primer 2-1 (SEQ ID NO: 15);

a third probe: an ORF1ab probe 2-1 (SEQ ID NO: 16).

a third composition:

a first primer pair: an N gene forward primer 1 (SEQ ID NO: 5) and an N gene reverse primer 1 (SEQ ID NO: 6);

a first probe: an N gene probe 1 (SEQ ID NO: 7);

a second primer pair: an ORF1ab forward primer 1-1 (SEQ ID NO: 8) and an ORF1ab reverse primer 1-1 (SEQ ID NO: 9);

a second probe: an ORF1ab probe 1-1 (SEQ ID NO: 10); and

a third primer pair: an ORF1ab forward primer 2-2 (SEQ ID NO: 17) and an ORF1ab reverse primer 2-2 (SEQ ID NO: 18);

a third probe: an ORF1ab probe 2-2 (SEQ ID NO: 19).

a fourth composition:

a first primer pair: an N gene forward primer 1 (SEQ ID NO: 5) and an N gene reverse primer 1 (SEQ ID NO: 6);

a first probe: an N gene probe 1 (SEQ ID NO: 7);

a second primer pair: an ORF1ab forward primer 1-2 (SEQ ID NO: 11) and an ORF1ab reverse primer 1-2 (SEQ ID NO: 12);

a second probe: an ORF1ab probe 1-2 (SEQ ID NO: 13); and

a third primer pair: an ORF1ab forward primer 2-1 (SEQ ID NO: 14) and an ORF1ab reverse primer 2-1 (SEQ ID NO: 15);

a third probe: an ORF1ab probe 2-1 (SEQ ID NO: 16).

Each composition further has an internal standard upstream primer, an internal standard downstream primer, and an internal standard probe (SEQ ID NOs: 20-22).

Moreover, the fluorescent reporter group of the first probe is ROX, the fluorescent reporter groups of the second and third probes are FAM, and the fluorescent reporter group of the internal standard probe is HEX. The 3′ end of the probe also has a quenching group such as BHQ1 or BHQ2.

### Example 2. Method for Detecting SARS-CoV-2

A sample for detection in the present invention is a throat swab, sputum, bronchoalveolar lavage fluid, or blood. The following operations are carried out:

(1) mixing a nucleic acid release agent S1014 of Sansure Biotech with a positive control at a ratio of 1:1, and lysing at room temperature for 10 minutes;

(2) according to the proportions of 26 μL of each reaction solution and 4 μL of mixed enzyme, taking a corresponding amounts of reaction solution and mixed enzyme, mixing well, and dispensing into each reaction tube at a volume of 30 μL per reaction; adding 20 μL of the lysed to-be-tested sample to each reaction tube, then covering with a PCR tube cap, instantaneously centrifuging, and placing it in a real-time fluorescent PCR instrument; and

(3) performing reaction and detection under the following cycle conditions (a FAM channel, an ROX channel and a HEX channel are selected for fluorescence acquisition):

qPCR procedure

| | | | | Number |
|---|---|---|---|---|
| | | | | of |
| Step | | Temperature | Time | cycles |
| 1 | Reverse transcription | 60° C. | 15 minutes | 1 |
| 2 | cDNA pre-denaturation | 95° C. | 1 minute | 1 |
| 3 | Denaturation | 95° C. | 15 seconds | 45 |
| | Annealing, extension | 60° C. | 20 seconds | |
| | and fluorescence | | | |
| | acquisition | | | |
| 4 | Instrument cooling | 25° C. | 10 seconds | 1 |

After the reaction is completed, the results are automatically saved, and the amplification curves of the detection standard and the internal standard are analyzed respectively. The Start value, End value and Threshold value of Baseline are adjusted according to the image after analysis (a user can adjust it according to the actual situation, the Start value can be set between 3 and 15, and the End value can be set between 5 and 20, the amplification curve of the negative control is adjusted to be straight or below a threshold line), Analyze is clicked to analyze, so that the parameters meet the requirements in the following “Quality Control”, and then the qualitative results are recorded in a Plate window.

(4) Quality Control

SARS-CoV-2-PCR-negative control: the FAM, ROX and internal standard (HEX) channels have no Ct value or Ct>40;

SARS-CoV-2-PCR-positive control: the FAM, ROX and internal standard (HEX) channels all have Ct≤35.

The requirements above should all be met in the same experiment, otherwise, the experiment is invalid and needs to be repeated.

### Example 3. Composition of the Present Invention for Detecting SARS-CoV-2

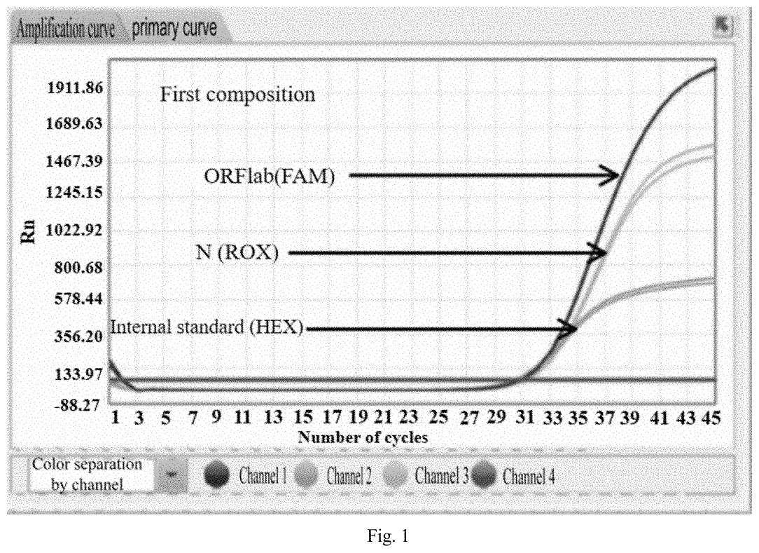

The preservation samples of novel coronavirus from the CDC of China are tested using the first composition in Example 1 of the present invention according to the method described in Example 2, and the experiment is repeated twice, and the results are as shown in FIG. 1.

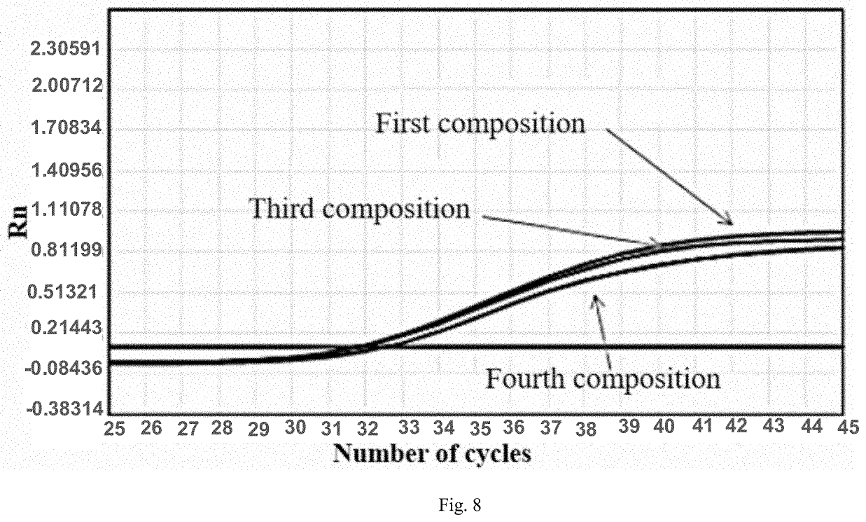

The preservation samples of the novel coronavirus from the CDC of China are tested using the first composition, the second composition, and the third composition in Example 1 of the present invention according to the method described in Example 2. FIG. 8 shows the detection result of the FAM channel.

As can be seen from FIG. 1 and FIG. 8, each amplification curve is tall and straight, and the Ct value meets the conditions, indicating that the compositions of the present invention can detect SARS-CoV-2.

### Example 4. Comparison of the Detection Effects of the Composition of the Present Invention and a Single-Primer Probe Composition of ORF1ab

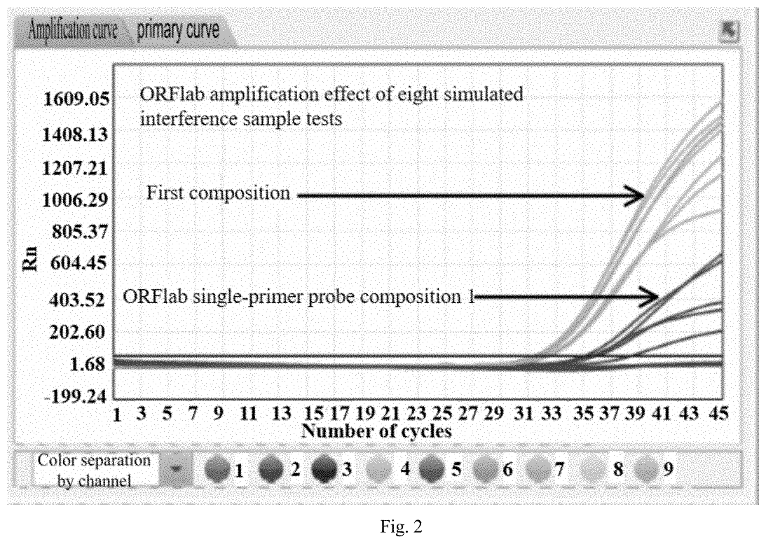

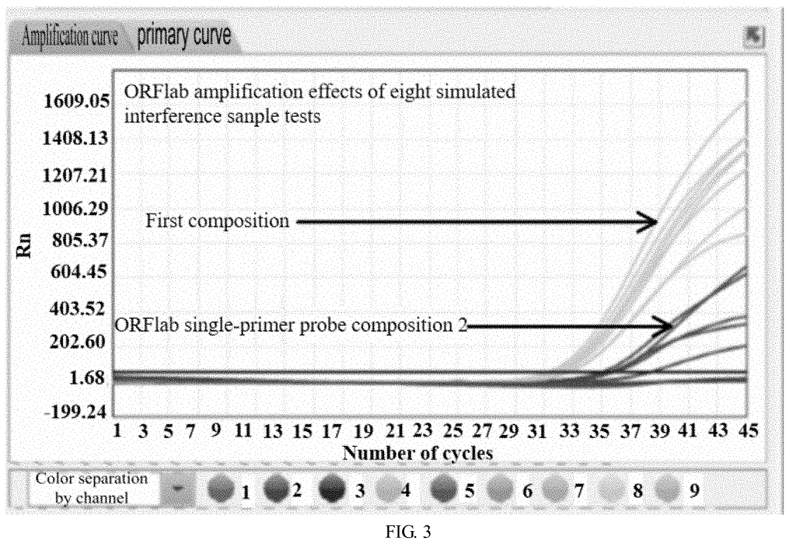

Eight cases of novel coronavirus samples with negative cotton throat swab samples stored in a preservation solution for more than one week and positive nucleic acid added as simulated interference samples are tested using the first composition described in Example 1 of the present invention, an ORF1ab single-primer probe composition 1 (excluding SEQ ID NOs: 14-16, the rest of the composition is consistent with the first composition), and an ORF1ab single-primer probe combination 2 (excluding SEQ ID NOs: 8-10, the rest of the composition is consistent with the first composition) according to the method described in Example 2. The test results are as shown in FIG. 2 and FIG. 3.

As can be seen from FIG. 2 and FIG. 3, the Ct value of the first composition of the present invention containing ORF1ab double primers is significantly moved forward than that of the single-primer probe composition, indicating that the sensitivity is higher than that of the single-primer probe composition.

### Example 5. Comparison of the Detection Effects of the Composition of the Present Invention and the Primer Probes of the CDC of China, the CDC of the United States, and the University of Hong Kong

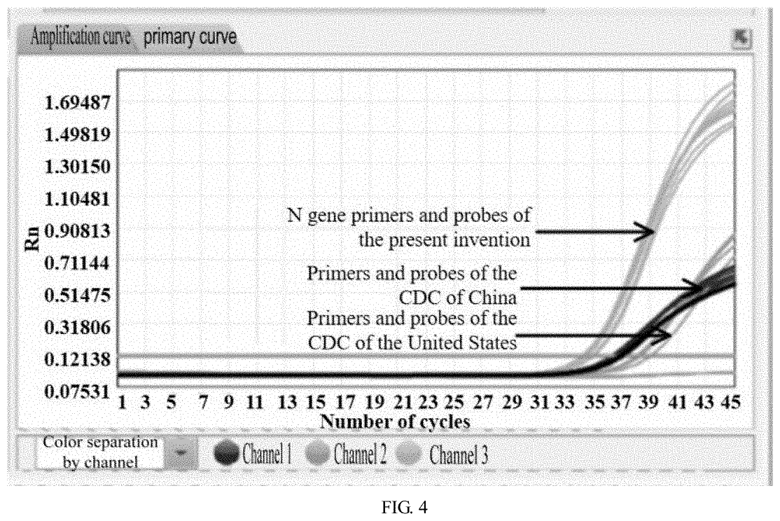

Combined detection is performed on 5-10 copies/ml of the novel coronavirus sample (a virus culture solution) by using the first primer pair and the first probe (SEQ ID NOs: 5-7) in the composition described in Example 1 of the present invention, the primer probes (three sets) of the CDC of China and the CDC of the United States (because the CDC of the United States only provides the primers and probes of the N gene, this example can only compare the difference of the N gene) according to the method described in Example 2, and the detection is repeated 10 times. The detection results are as shown in FIG. 4. As can be seen from the figure, for the novel coronavirus of the same concentration, the Ct value of the N gene detected by the first primer pair and the first probe of the present invention is significantly moved forward than that of the primer probes of the CDC of China and the CDC of the United States.

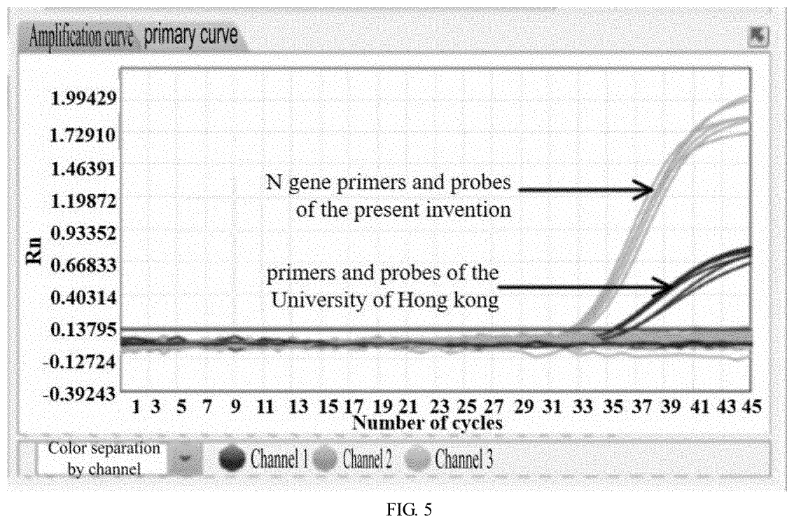

Combined detection is performed on 5-10 copies/ml of the novel coronavirus sample (the bronchoalveolar lavage fluid sample, the virus culture solution, the plasma sample) using the first primer pair and the first probe in the composition described in Example 1 of the present invention, and the primers and probes of the University of Hong Kong according to the method described in Example 2, and the detection is repeated 10 times. The detection results are as shown in FIG. 5. As can be seen from the figure, for the novel coronavirus of the same concentration, the Ct value of the N gene detected by the first primer pair and the first probe of the present invention is significantly moved forward than that of the primer probes of the University of Hong Kong.

SARS-CoV-2 can be detected more accurately by using the composition of the present invention.

The specific sequences of various primers and probes used as comparisons in this example are given below:

China CDC SARS-CoV-2 N upstream primer:
(SEQ ID NO: 23)
5′-GGGGAACTTCTCCTGCTAGAAT-3′;
China CDCSARS-CoV-2 N downstream primer:
(SEQ ID NO: 24)
5′-CAGACATTTTGCTCTCAAGCTG-3′;
China CDCSARS-CoV-2 N probe:
(SEQ ID NO: 25)
5′-TTGCTGCTGCTTGACAGATT-3′;
US CDCSARS-CoV-2_N1 upstream primer:
(SEQ ID NO: 26)
5′-GACCCCAAAATCAGCGAAAT-3′;
US CDCSARS-CoV-2_N1 downstream primer:
(SEQ ID NO: 27)
5′-TCTGGTTACTGCCAGTTGAATCTG-3′;
US CDCSARS-CoV-2_N1 probe:
(SEQ ID NO: 28)
5′-ACCCCGCATTACGTTTGGTGGACC-3′;
US CDCSARS-CoV-2_N2 upstream primer:
(SEQ ID NO: 29)
5′-TTACAAACATTGGCCGCAAA-3′;
US CDCSARS-CoV-2_N2 downstream primer:
(SEQ ID NO: 30)
5′-GCGCGACATTCCGAAGAA-3′;
US CDCSARS-CoV-2_N2 probe:
(SEQ ID NO: 31)
5′-ACAATTTGCCCCCAGCGCTTCAG-3′;
US CDCSARS-CoV-2_N3 upstream primer:
(SEO ID NO: 32)
5′-GGGAGCCTTGAATACACCAAAA-3′;
US CDCSARS-CoV-2_N3 downstream primer:
(SEQ ID NO: 33)
5′-TGTAGCACGATTGCAGCATTG-3′;
US CDCSARS-CoV-2_N3 probe:
(SEQ ID NO: 34)
5′-AYCACATTGGCACCCGCAATCCTG-3′;
HKU-N upstream primer:
(SEQ ID NO: 35)
5′-TAATCAGACAAGGAACTGATTA-3′;
HKU-N downstream primer:
(SEQ ID NO: 36)
5′-CGAAGGTGTGACTTCCATG-3′;
HKU-N probe:
(SEQ ID NO: 37)
5′-FAM-GCAAATTGTGCAATTTGCGG-TAMRA-3′.

### Example 6. Anti-Interference Test of the Composition of the Present Invention

The novel coronavirus samples with interfering substances are verified using the first composition (SEQ ID NOs: 5-10, 14-16, and 20-22) in Example 1 of the present invention according to the method described in Example 2.

Upon test and verification, the interfering substances do not significantly interfere with the test results of the kit. The experimental results are as shown in Table 1, and no interference occurs in the three repeated experiments.

TABLE 1

| | | Result (three | | | Result (three |
|---|---|---|---|---|---|
| Interfering | | repeated | Interfering | | repeated |
| substances | Concentration | experiments) | substances | Concentration | experiments) |
| Purified mucin | 20 pg/mL | 0/3 | Dexamethasone | 50 μg/mL | 0/3 |
| Human whole | 5% (v/v) | 0/3 | Cefmenoxime | 50 μg/mL | 0/3 |
| blood | | | hydrochloride | | |
| Oxymetazoline | 100 μg/mL | 0/3 | Oseltamivir | 100 μg/mL | 0/3 |
| hydrochloride | | | | | |
| Beclomethasone | 50 μg/mL | 0/3 | Zanamivir | 100 μg/mL | 0/3 |
| Ribavirin | 100 μg/mL | 0/3 | Azithromycin | 100 μg/mL | 0/3 |
| Fluticasone | 200 μg/mL | 0/3 | Budesonide | 320 μg/mL | 0/3 |
| α-interferon | 300 μg/mL | 0/3 | Phenylephrine | 125 μg/mL | 0/3 |
| Mometasone | 100 μg/mL | 0/3 | Tobramycin | 100 μg/mL | 0/3 |
| Ritonavir | 100 μg/mL | 0/3 | Flunisolide | 100 μg/mL | 0/3 |
| Histamine | 200 μg/mL | 0/3 | Peramivir | 100 μg/mL | 0/3 |
| hydrochloride | | | | | |
| Lopinavir | 100 μg/mL | 0/3 | Mupirocin | 100 μg/mL | 0/3 |
| Triamcinolone | 100 μg/mL | 0/3 | Anhydrous | 20% (v/v) | 0/3 |
| acetonide | | | ethanol | | |
| Abidore | 100 μg/mL | 0/3 | Sodium | 60 μg/mL | 0/3 |
| | | | chloride | | |
| Urea | 100 μg/mL | 0/3 | Heme | 10 μg/mL | 0/3 |

### Example 7. Specificity Verification of the Composition of the Present Invention

The first composition (SEQ ID NOs: 5-10, 14-16, and 20-22) of the present invention does not cross-react with the positive samples such as coronaviruses (NL63, HKU1, 229E, OC43), SARS coronavirus, MERS coronavirus, influenza A virus, influenza B Yamagata, Victoria, influenza A H1N1, influenza A H3N2, influenza A H5N1, influenza A H7N9, respiratory syncytial virus types A and B, rhinovirus types A, B and C, adenovirus types 1, 2, 3, 4, 5, 7 and 55, parainfluenza virus types 1, 2, and 3, enterovirus types A and B, enterovirus C (EV-C95), enterovirus D (EV-D70), metapneumovirus, human metapneumovirus, Cryptococcus neoformans, Streptococcus pyogenes, Acinetobacter baumannii, Pneumocystis, Klebsiella pneumoniae, Streptococcus pneumoniae, Haemophilus influenzae, Pseudomonas aeruginosa, Legionella pneumophila, Bacillus pertussis, Staphylococcus aureus, Mycoplasma pneumoniae, Streptococcus pneumoniae, Klebsiella pneumoniae, Chlamydia pneumoniae, Epstein-Barr virus, human cytomegalovirus, Aspergillus fumigatus, Candida albicans, Candida glabrata, Mycobacterium tuberculosis, nontuberculous Mycobacterium, norovirus, rotavirus, aquatic sore virus, measles virus, mumps virus, and human genome DNA.

The test results for some specific examples are listed in Table 2 below.

TABLE 2

| | | | | Ct value |
|---|---|---|---|---|
| | | Source/sample | | (ORF1ab |
| Positive samples | Strain | type | Concentration | gene/N gene) |
| Human coronavirus | 229E | Clinical | 1.0 × 10⁶ copies/ml | Not detected/not |
| 229E | | sample | | detected |
| Human coronavirus | OC43 | Clinical | 1.0 × 10⁶ copies/ml | Not detected/not |
| OC43 | | sample | | detected |
| Human coronavirus | HKU1 | Clinical | 1.0 × 10⁶ copies/ml | Not detected/not |
| HKU1 | | sample | | detected |
| Human coronavirus | NL63 | Clinical | 1.0 × 10⁶ copies/ml | Not detected/not |
| NL63 | | sample | | detected |
| SARS-coronavirus | | RNA | 1.0 × 10⁶ copies/ml | Not detected/not |
| | | | | detected |
| MERS-coronavirus | | Pseudovirus | 1.0 × 10⁶ copies/ml | Not detected/not |
| | | | | detected |
| Adenovirus 1 | | Clinical | 1.0 × 10⁶ copies/ml | Not detected/not |
| | | sample | | detected |
| Human | | Clinical | 1.0 × 10⁶ copies/ml | Not detected/not |
| Metapneumovirus | | sample | | detected |
| (hMPV) | | | | |
| Parainfluenza | | Clinical | 1.0 × 10⁶ copies/ml | Not detected/not |
| vims 1-4 | | sample | | detected |
| Influenza A | | National | 1.0 × 10⁶ TCID50/L | Not detected/not |
| | | standard | | detected |
| Influenza B | | Clinical | 1.0 × 10⁶ copies/ml | Not detected/not |
| | | sample | | detected |
| Enterovirus (EV- | | Clinical | 1.0 × 10⁶ copies/ml | Not detected/not |
| C95) | | sample | | detected |
| Respiratory | | Clinical | 1.0 × 10⁶ copies/ml | Not detected/not |
| syncytial virus | | sample | | detected |
| Rhinovirus | | Clinical | 1.0 × 10⁶ copies/ml | Not detected/not |
| | | sample | | detected |
| Chlamydia | | Clinical | 1.0 × 10⁶ copies/ml | Not detected/not |
| pneumonia | | sample | | detected |
| Haemophilus | | National | 1.0 × 10⁶ CFU/ml | Not detected/not |
| influenzae | | standard | | detected |
| Legionella | | National | 1.0 × 10⁶ CFU/ml | Not detected/not |
| pneumophila | | standard | | detected |
| Mycobacterium | | Clinical | 1.0 × 10⁶ copies/ml | Not detected/not |
| tuberculosis | | sample | | detected |
| Streptococus | | National | 1.0 × 10⁶ CFU/ml | Not detected/not |
| pneumonia | | standard | | detected |
| Streptococcus | | Clinical | 1.0 × 10⁶ copies/ml | Not detected/not |
| pyogenes | | sample | | detected |
| Bordetella pertussis | | Clinical | 1.0 × 10⁶ copies/ml | Not detected/not |
| | | sample | | detected |
| Mycoplasma | | Clinical | 1.0 × 10⁶ copies/ml | Not detected/not |
| pneumoniae | | sample | | detected |
| Pneumocystis | | Clinical | 1.0 × 10⁶ copies/ml | Not detected/not |
| jirovecii (PJP) | | sample | | detected |

### Example 8. Precision Verification of the Composition of the Present Invention

In this experiment, SARS-CoV-2 positive samples are diluted to medium concentration positive and critical concentration positive, and SARS-CoV-2 negative samples are added as to-be-tested samples for intra-batch imprecision determination.

For each precision sample, one experimental batch is analyzed per day, and each precision sample is repeated five times for five consecutive days. A qualified novel coronavirus detection kit (including the first composition of the present invention (SEQ ID NOs: 5-10, 14-16 and 20-22)) is used for detection on the same SLAN 96P instrument, the positive detection rate and negative detection rate are analyzed, and the precision performance of the kit are investigated. The results are as shown in Table 3 below. The results show that the detection results of the composition of the present invention are consistent in each batch, reflecting good precision.

TABLE 3
Statistics of Ct values of reagent intra-batch imprecision test results

| | | Medium | | Critical | | | |
|---|---|---|---|---|---|---|---|
| | | concentration | | concentration | | | |
| Number | Serial | positive | | positive | | Negative | |
| of days | number | ORF1ab | N | ORF1ab | N | ORF1ab | N |
| Day 1 | 1 | 26.30 | 25.48 | 32.03 | 32.03 | No Ct | No Ct |
| | 2 | 26.31 | 25.45 | 32.04 | 32.32 | No Ct | No Ct |
| | 3 | 26.29 | 25.51 | 31.79 | 32.37 | No Ct | No Ct |
| | 4 | 26.31 | 25.47 | 32.18 | 32.06 | No Ct | No Ct |
| | 5 | 26.31 | 25.54 | 31.88 | 32.28 | No Ct | No Ct |
| Day 2 | 6 | 26.40 | 25.58 | 31.53 | 32.42 | No Ct | No Ct |
| | 7 | 26.34 | 25.54 | 31.85 | 32.40 | No Ct | No Ct |
| | 8 | 26.36 | 25.65 | 31.73 | 32.29 | No Ct | No Ct |
| | 9 | 26.31 | 25.53 | 32.23 | 32.22 | No Ct | No Ct |
| | 10 | 26.30 | 25.54 | 32.06 | 32.14 | No Ct | No Ct |
| Day 3 | 11 | 26.37 | 25.39 | 31.88 | 32.24 | No Ct | No Ct |
| | 12 | 26.36 | 25.48 | 32.22 | 32.20 | No Ct | No Ct |
| | 13 | 26.29 | 25.47 | 32.24 | 32.30 | No Ct | No Ct |
| | 14 | 26.28 | 25.50 | 32.07 | 32.10 | No Ct | No Ct |
| | 15 | 26.33 | 25.43 | 32.27 | 32.03 | No Ct | No Ct |
| Day 4 | 16 | 25.14 | 25.40 | 31.99 | 32.19 | No Ct | No Ct |
| | 17 | 25.16 | 25.41 | 32.17 | 32.10 | No Ct | No Ct |
| | 18 | 25.15 | 25.51 | 31.76 | 32.87 | No Ct | No Ct |
| | 19 | 25.17 | 25.44 | 32.03 | 32.39 | No Ct | No Ct |
| | 20 | 25.18 | 25.41 | 32.52 | 32.91 | No Ct | No Ct |
| Day 5 | 21 | 25.16 | 25.39 | 32.10 | 32.21 | No Ct | No Ct |
| | 22 | 25.14 | 25.48 | 32.03 | 32.01 | No Ct | No Ct |
| | 23 | 25.13 | 25.45 | 31.85 | 31.92 | No Ct | No Ct |
| | 24 | 25.13 | 25.41 | 31.97 | 32.26 | No Ct | No Ct |
| | 25 | 25.17 | 25.45 | 32.06 | 32.67 | No Ct | No Ct |
| | Positive | 100% | 100% | 100% | 100% | 0% | 0% |
| | detection | | | | | | |
| | rate | | | | | | |

### Example 9. Detection of Comparative Example Composition of the ORF1ab Gene of the Present Invention

To further demonstrate the advantages of the specific areas mentioned in the present invention, the inventors use anther specific area on the ORF1ab gene:

ATAAAGAAATGTATCTAAAGTTGCGTAGTGATGTGCTATTACCTCTTACGCAAT ATAATAGATACTTAGCTCTTTATAATAAGTACAAGTATTTTAGTGGAGCAATGGATAC AACTAGCTACAGAGAAGCTGCTTGTTGTCATCTCGCAAAGGCTCTCAATGACTTCA GTAACTCAGGTTCTGATGTTCTTTA (SEQ ID NO: 38), and on this basis, the primer and the probe are designed, and the specific sequence thereof is as shown below:

an ORF1ab forward primer 3:
(SEQ ID NO: 39)
5′-ATGTATCTAAAGTTGCGTAG-3′;
an ORF1ab reverse primer 3:
(SEQ ID NO 40)
5′-GTTACTGAAGTCATTGAGAGCCT-3′;
an ORF1ab probe 3:
(SEQ ID NO: 41)
5′-ATGTGCTATTACCTCTTACGCAATATAAT-3′

First, the inventors verify the detection efficiency of primers and probes using one ORF1ab gene detection alone.

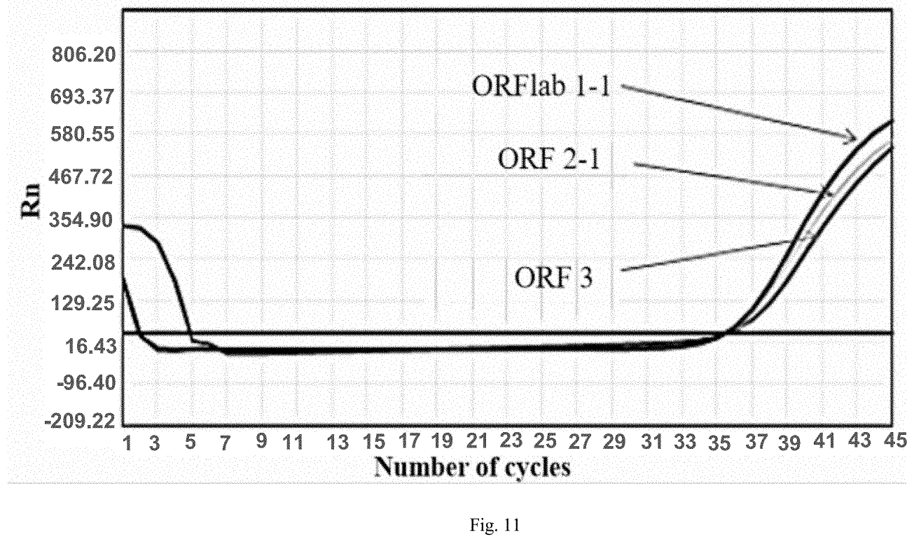

The ORF1ab gene is detected by respectively using combinations of three primers and probes: 1. an ORF1ab forward primer 1-1, an ORF1ab reverse primer 1-1, and an ORF1ab probe 1-1; 2. an ORF1ab forward primer 2-1, an ORF1ab reverse primer 2-1, and an ORF1ab probe 2-1; and 3. an ORF1ab forward primer 3, an ORF1ab reverse primer 3, and an ORF1ab probe 3, and the results are as shown in FIG. 11. The results show that the detection efficiency of the three primers and probes on the ORF1ab gene is comparable. That is, the detection effect of a system designed for SEQ ID NO: 38 alone is comparable to that of a system designed for SEQ ID NOs: 1 and 2 alone.

SEQ ID NOs: 39-41 (the ORF1ab forward primer 3, the ORF1ab reverse primer 3, and the ORF1ab probe 3) are combined with SEQ ID NOs: 8-10 mentioned in Example 1 of the present invention to form a fifth composition. SEQ ID NOs: 39-41 (the ORF1ab forward primer 3, the ORF1ab reverse primer 3, and the ORF1ab probe 3) are combined with SEQ ID NOs: 14-16 mentioned in the example of the present invention to form a sixth composition.

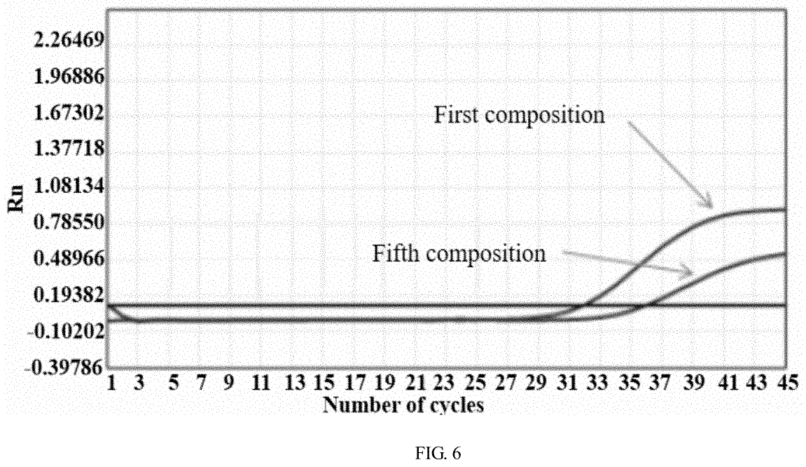

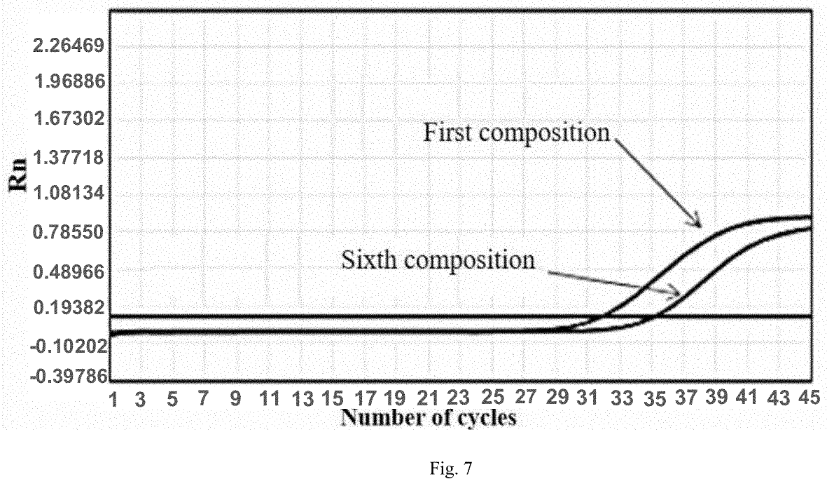

The fifth composition and the sixth composition respectively implement detection according to the method in Example 2, and the detection results of the FAM channel are as shown in FIG. 6 and FIG. 7, respectively. Although in the above-mentioned individual system verification, it is found that the detection effects of the individual primer probes are comparable (as shown in FIG. 11), after the combination, it is found that the Ct values of the fifth composition and the sixth composition are significantly moved backward than that of ORF1ab of the first composition of the present invention. Therefore, it is proved that the combination of specific areas of ORF1ab found by the present invention has the advantage of higher sensitivity in detection.

### Example 10. Detection of Comparative Example Composition of the N Gene of the Present Invention

To further demonstrate the advantages of the specific areas mentioned in the present invention, the inventors first design an N gene primer probe combination 2 for the above-mentioned specific area of the N gene (SEQ ID NO: 3), and the specific sequence thereof is as shown below:

an N gene forward primer 2:
(SEQ ID NO: 43)
5′-ACTACCTAGGAACTGGGCCAG-3′;
an N gene reverse primer 2:
(SEQ ID NO: 44)
5′-AGCAGGATTGCGGGTGCCAAT-3′;
and
an N gene probe 2:
(SEQ ID NO: 45)
5′-ACTTCCCTATGGTGCTAACAAAGACGGCAT-3′.

Secondly, the inventors find another specific area on the N gene:

TGAGAGCAAAATGTCTGGTAAAGGCCAACAACAACAAGGCCAAACTGTCACT AAGAAATCTGCTGCTGAGGCTTCTAAGAAGCCTCGGCAAAAACGTACTGCCACTA AAGCATACAATGTAACACAAGCTTTCGGCAGACGTGGTCCAGAACAAACCCAAG GAAATTTTGGGGACCAGGAACTAATCAGACAAGGAACTGATTACAAACATTGGCC GCA (SEQ ID NO: 42), and on this basis, an N gene primer probe combination 3 is designed, and the specific sequence thereof is as shown below:

an N gene forward primer 3:
(SEQ ID NO: 46)
5′-ATGTCTGGTAAAGGCCAACAAC-3′;
an N gene reverse primer 3:
(SEQ ID NO: 47)
5′-AGTTCCTTGTCTGATTAGTTC-3′;
and
an N gene probe 3:
(SEQ ID NO: 48)
5′-ACTGTCACTAAGAAATCTGCTGCTGAGGC-3′.

First, the inventors verify the detection efficiency of the primer probe combination using one N gene detection alone.

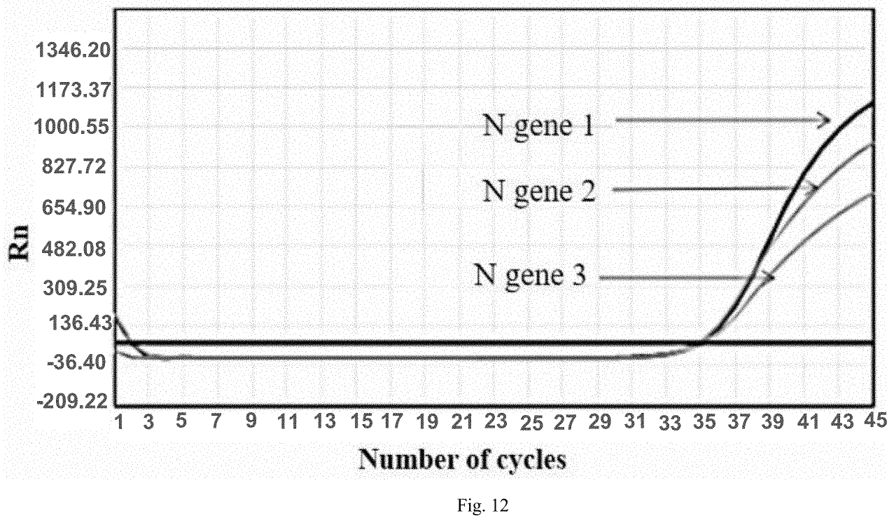

The N gene is detected by respectively using combinations of three primers and probes: 1. an N gene forward primer 1, an N gene reverse primer 1, and an N gene probe 1; 2. an N gene forward primer 2, an N gene reverse primer 2, and an N gene probe 2; and 3. an N gene forward primer 3, an N gene reverse primer 3, and an N gene probe 3, and the results are as shown in FIG. 12. The results show that the detection efficiency of the combinations of the three primers and probes on the N gene is comparable. That is, the detection effect of a system designed for SEQ ID NO: 42 alone is comparable to that of a system designed for SEQ ID NO: 3 alone.

The N gene primer probe combination 2 is combined with SEQ ID NOs: 8-10 and SEQ ID NOs: 14-16 mentioned in Example 1 of the present invention to form a seventh composition. The N gene primer probe combination 3 is combined with SEQ ID NOs: 8-10 and SEQ ID NOs: 14-16 mentioned in the example of the present invention to form an eighth composition.

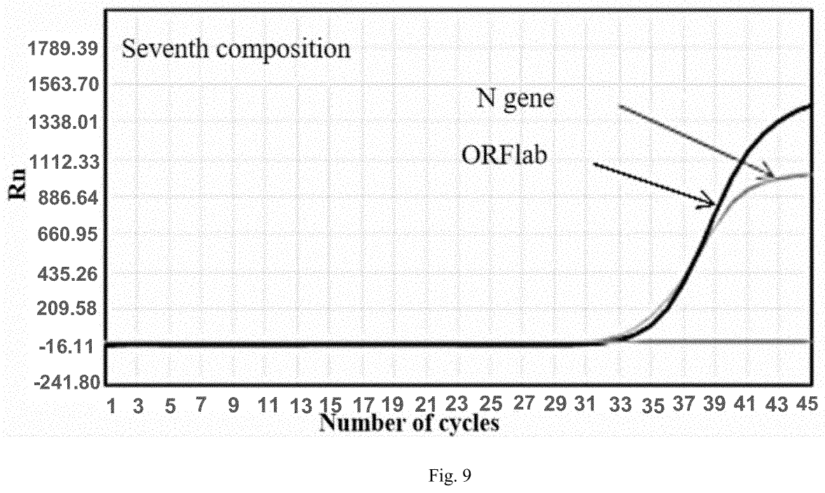

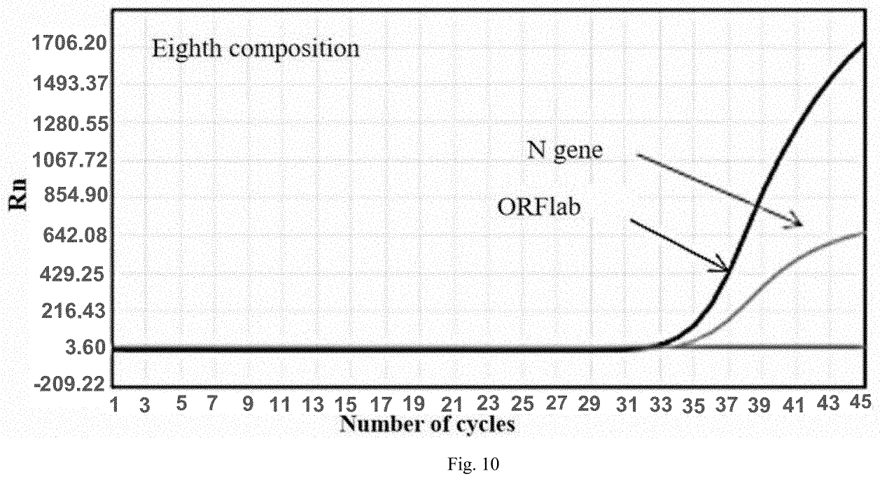

The seventh composition and the eighth composition respectively implement detection according to the method in Example 2, and the detection results of the ROX channel are as shown in FIG. 9 and FIG. 10, respectively. Although in the above-mentioned individual system verification, it is found that the detection effects of the individual primer probes are comparable (as shown in FIG. 12), after the combination, it is found that the detection efficiency of the seventh composition is comparable to that of the first composition, and the Ct value of the eighth composition is significantly moved backward than that of N gene of the first composition of the present invention. Therefore, it is proved that the specific area of the N gene found by the present invention has the advantage of higher sensitivity in combined detection.

What is claimed is:

1. A composition for detecting SARS-CoV-2, comprising:
a first primer pair, used for specifically amplifying an N gene of SARS-CoV-2;
a first probe, used for detecting an amplification product of the first primer pair;
a second primer pair, used for specifically amplifying an area as shown in SEQ ID NO: 1 in an ORF1ab gene;
a second probe, used for detecting an amplification product of the second primer pair;
a third primer pair, used for specifically amplifying an area as shown in SEQ ID NO: 2 in the ORF1ab gene; and
a third probe, used for detecting an amplification product of the third primer pair.

2. The composition according to claim 1, wherein the second primer pair and the second probe are:
an ORF1ab forward primer 1-1:
(SEQ ID NO: 8)
5′-ATTCACCTAATTTAGCATGGCCTC-3′;
an ORF1ab reverse primer 1-1:
(SEQ ID NO: 9)
5′-ACATCTGTCGTAGTGCAACAGGAC-3′;
and
an ORF1ab probe 1-1:
(SEQ ID NO: 10)
5′-TTGTAACAGCTTTAAGGGCCAATTCTGCTGT-3′;
or
an ORF1ab forward primer 1-2:
(SEQ ID NO: 11)
5′-ATGGCCTCTTATTGTAACAGCT-3′;
an ORF1ab reverse primer 1-2:
(SEQ ID NO: 12)
5′-AGCAGTTTGTGTAGTACCGGCAGC-3′;
and
an ORF1ab probe 1-2:
(SEQ ID NO: 13)
5′-CCAATTCTGCTGTCAAATTACAGAATA-3′.

3. The composition according to claim 1, wherein the third primer pair and the third probe are:
an ORF1ab forward primer 2-1:
(SEQ ID NO: 14)
5′-GAAGGTTCTGTTGCTTATGAAAGTT-3′;
an ORF1ab reverse primer 2-1:
(SEQ ID NO: 15)
5′-TACCACTCTAACAGAACCTTC-3′;
and
an ORF1ab probe 2-1:
(SEQ ID NO: 16)
5′-GCCCTGACACACGTTATGTGCTCATGGA-3′;
or
an ORF1ab forward primer 2-2:
(SEQ ID NO: 17)
5′-ATGAAAGTTTACGCCCTGAC-3′;
an ORF1ab reverse primer 2-2:
(SEQ ID NO: 18)
5′-CCGTGCCTACAGTACTCAGAATC-3′;
and
an ORF1ab probe 2-2:
(SEQ ID NO: 19)
5′-ATGTGCTCATGGATGGCTCTATTATTCAATT-3′.

4. The composition according to claim 1, wherein the first primer pair is used for specifically amplifying an area as shown in SEQ ID NO: 3 in the N gene.

5. The composition according to claim 4, wherein the first primer pair and the first probe are:
an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
an N gene probe 1:
(SEQ ID NO: 7)
5′-GCCAGAAGCTGGACTTCCCTATGGTGC-3′;
or
an N gene forward primer 2:
(SEQ ID NO: 43)
5′-ACTACCTAGGAACTGGGCCAG-3′;
an N gene reverse primer 2:
(SEQ ID NO: 44)
5′-AGCAGGATTGCGGGTGCCAAT-3′;
and
an N gene probe 2:
(SEQ ID NO: 45)
5′-ACTTCCCTATGGTGCTAACAAAGACGGCAT-3′.

6. The composition according to claim 1, the composition further comprises:
a first primer pair:
an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
a first probe:
an N gene probe 1:
(SEQ ID NO: 7)
5′-GCCAGAAGCTGGACTTCCCTATGGTGC-3′;
a second primer pair:
an ORF1ab forward primer 1-1:
(SEQ ID NO: 8)
5′-ATTCACCTAATTTAGCATGGCCTC-3′;
an ORF1ab reverse primer 1-1:
(SEQ ID NO: 9)
5′-ACATCTGTCGTAGTGCAACAGGAC-3′;
and
a second probe:
an ORF1ab probe 1-1:
(SEQ ID NO: 10)
5′-TTGTAACAGCTTTAAGGGCCAATTCTGCTGT-3′;
and
a third primer pair:
an ORF1ab forward primer 2-1:
(SEQ ID NO: 14)
5′-GAAGGTTCTGTTGCTTATGAAAGTT-3′;
an ORF1ab reverse primer 2-1:
(SEQ ID NO: 15)
5′-TACCACTCTAACAGAACCTTC-3′;
and
a third probe:
an ORF1ab probe 2-1:
(SEQ ID NO: 16)
5′-GCCCTGACACACGTTATGTGCTCATGGA-3′.

7. The composition according to claim 1, the composition further comprises:
a first primer pair:
an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
a first probe:
an N gene probe 1:
(SEQ ID NO: 7)
5′-GCCAGAAGCTGGACTTCCCTATGGTGC-3′;
a second primer pair:
an ORF1ab forward primer 1-2:
(SEQ ID NO: 11)
5′-ATGGCCTCTTATTGTAACAGCT-3′;
an ORF1ab reverse primer 1-2:
(SEQ ID NO: 12)
5′-AGCAGTTTGTGTAGTACCGGCAGC-3′;
and
a second probe:
an ORF1ab probe 1-2:
(SEQ ID NO: 13)
5′-CCAATTCTGCTGTCAAATTACAGAATA-3′;
and
a third primer pair:
an ORF1ab forward primer 2-2:
(SEQ ID NO: 17)
5′-ATGAAAGTTTACGCCCTGAC-3′;
an ORF1ab reverse primer 2-2:
(SEQ ID NO: 18)
5′-CCGTGCCTACAGTACTCAGAATC-3′;
and
a third probe:
an ORF1ab probe 2-2:
(SEQ ID NO: 19)
5′-ATGTGCTCATGGATGGCTCTATTATTCAATT-3′.

8. The composition according to claim 1, the composition further comprises:
a first primer pair:
an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
a first probe:
an N gene probe 1:
(SEQ ID NO: 7)
5′-GCCAGAAGCTGGACTTCCCTATGGTGC-3′;
a second primer pair:
an ORF1ab forward primer 1-1:
(SEQ ID NO: 8)
5′-ATTCACCTAATTTAGCATGGCCTC-3′;
an ORF1ab reverse primer 1-1:
(SEQ ID NO: 9)
5′-ACATCTGTCGTAGTGCAACAGGAC-3′;
and
a second probe:
an ORF1ab probe 1-1:
(SEQ ID NO: 10)
5′-TTGTAACAGCTTTAAGGGCCAATTCTGCTGT-3′;
and
a third primer pair:
an ORF1ab forward primer 2-2:
(SEQ ID NO: 17)
5′-ATGAAAGTTTACGCCCTGAC-3′;
an ORF1ab reverse primer 2-2:
(SEQ ID NO: 18)
5′-CCGTGCCTACAGTACTCAGAATC-3′;
and
a third probe:
an ORF1ab probe 2-2:
(SEQ ID NO: 19)
5′-ATGTGCTCATGGATGGCTCTATTATTCAATT-3′.

9. The composition according to claim 1, the composition further comprises:
a first primer pair:
an N gene forward primer 1:
(SEQ ID NO: 5)
5′-GGTATTTCTACTACCTAGGAACT-3′;
an N gene reverse primer 1:
(SEQ ID NO: 6)
5′-TGCAGCATTGTTAGCAGGATTG-3′;
and
a first probe:
an N gene probe 1:
(SEQ ID NO: 7)
5′-GCCAGAAGCTGGACTTCCCTATGGTGC-3′;
a second primer pair:
an ORF1ab forward primer 1-2:
(SEQ ID NO: 11)
5′-ATGGCCTCTTATTGTAACAGCT-3′;
an ORF1ab reverse primer 1-2:
(SEQ ID NO: 12)
5′-AGCAGTTTGTGTAGTACCGGCAGC-3′;
and
a second probe:
an ORF1ab probe 1-2:
(SEQ ID NO: 13)
5′-CCAATTCTGCTGTCAAATTACAGAATA-3′;
and
a third primer pair:
an ORF1ab forward primer 2-1:
(SEQ ID NO: 14)
5′-GAAGGTTCTGTTGCTTATGAAAGTT-3′;
an ORF1ab reverse primer 2-1:
(SEQ ID NO: 15)
5′-TACCACTCTAACAGAACCTTC-3′;
and
a third probe:
an ORF1ab probe 2-1:
(SEQ ID NO: 16)
5′-GCCCTGACACACGTTATGTGCTCATGGA-3′.

10. The composition according to claim 1, further comprising an internal standard upstream primer, an internal standard downstream primer, and an internal standard probe for monitoring.

11. The composition according to claim 1, further comprising an internal standard primer pair, used for specifically amplifying an area of an RNaseP gene as shown in SEQ ID NO: 4; and
an internal standard probe, used for detecting an amplification product of the internal standard primer pair.

12. The composition according to claim 11, wherein the internal standard upstream primer is: 5′-GAGGTGGGACTTCAGCATGGC-3′ (SEQ ID NO: 20); the internal standard downstream primer is: 5′-TGTTTCTTTTCCTTAAAGTCAAC-3′ (SEQ ID NO: 21); and the internal standard probe is 5′-AGATTTGGACCTGCGAGCGGGTTCTGA-3′ (SEQ ID NO: 22).

13. The composition according to claim 1, wherein in the composition, the first probe, the second probe and the third probe carry fluorescent reporter groups that do not interfere with each other.

14. The composition according to claim 1, wherein a dosage of the primer in the composition is 25 nM-200 nM.

15. The composition according to claim 1, wherein a dosage of the probe in the composition is 10 nM-80 nM.

16. A kit for detecting SARS-CoV-2, comprising the composition of claim 1.

17. A method for detecting SARS-CoV-2, comprising the following steps:
1) extracting or releasing nucleic acids from a to-be-tested sample;
2) using the composition of claim 1 to perform fluorescent quantitative PCR on the nucleic acids obtained in step 1); and
3) obtaining and analyzing a result.

18. The method according to claim 17, wherein the sample is selected from a group consisting of a throat swab, sputum, a bronchoalveolar lavage fluid, and blood.


SEQUENCE LISTING (48 sequences)

SEQ ID NO: 1 - DNA, 170 bp, unidentified
  source 1..170: mol_type = genomic DNA; note = an area on an ORF1ab gene in the genome of SARS-CoV-2
acaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaac

SEQ ID NO: 2 - DNA, 170 bp, unidentified
  source 1..170: mol_type = genomic DNA; note = an area on an ORF1ab gene in the genome of SARS-CoV-2
actagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctg

SEQ ID NO: 3 - DNA, 152 bp, unidentified
  source 1..152: mol_type = genomic DNA; note = an area on a N gene in the genome of SARS-CoV-2
atggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcg

SEQ ID NO: 4 - DNA, 270 bp, unidentified
  source 1..270: mol_type = genomic DNA; note = an area of an RNaseP gene
gaattcggcacgaggtgggacttcagcatggcggtgtttgcagatttggacctgcgagcgggttctgacctgaaggctctgcgcggacttgtggagacagccgctcaccttggctattcagttgttgctatcaatcatatcgttgactttaaggaaaagaaacaggaaattgaaaaaccagtagctgtttctgaactcttcacaactttgccaattgtacagggaaaatcaagaccaattaaaattttaactagattaacaattattgtc

SEQ ID NO: 5 - DNA, 23 bp, synthetic construct
  source 1..23: mol_type = other DNA; note = an N gene forward primer 1
ggtatttctactacctaggaact

SEQ ID NO: 6 - DNA, 22 bp, synthetic construct
  source 1..22: mol_type = other DNA; note = an N gene reverse primer 1
tgcagcattgttagcaggattg

SEQ ID NO: 7 - DNA, 27 bp, synthetic construct
  source 1..27: mol_type = other DNA; note = an N gene probe 1
gccagaagctggacttccctatggtgc

SEQ ID NO: 8 - DNA, 24 bp, synthetic construct
  source 1..24: mol_type = other DNA; note = an ORF1ab forward primer 1-1
attcacctaatttagcatggcctc

SEQ ID NO: 9 - DNA, 24 bp, synthetic construct
  source 1..24: mol_type = other DNA; note = an ORF1ab reverse primer 1-1
acatctgtcgtagtgcaacaggac

SEQ ID NO: 10 - DNA, 31 bp, synthetic construct
  source 1..31: mol_type = other DNA; note = an ORF1ab probe 1-1
ttgtaacagctttaagggccaattctgctgt

SEQ ID NO: 11 - DNA, 22 bp, synthetic construct
  source 1..22: mol_type = other DNA; note = an ORF1ab forward primer 1-2
atggcctcttattgtaacagct

SEQ ID NO: 12 - DNA, 24 bp, synthetic construct
  source 1..24: mol_type = other DNA; note = an ORF1ab reverse primer 1-2
agcagtttgtgtagtaccggcagc

SEQ ID NO: 13 - DNA, 27 bp, synthetic construct
  source 1..27: mol_type = other DNA; note = an ORF1ab probe 1-2
ccaattctgctgtcaaattacagaata

SEQ ID NO: 14 - DNA, 25 bp, synthetic construct
  source 1..25: mol_type = other DNA; note = an ORF1ab forward primer 2-1
gaaggttctgttgcttatgaaagtt

SEQ ID NO: 15 - DNA, 21 bp, synthetic construct
  source 1..21: mol_type = other DNA; note = an ORF1ab reverse primer 2-1
taccactctaacagaaccttc

SEQ ID NO: 16 - DNA, 28 bp, synthetic construct
  source 1..28: mol_type = other DNA; note = an ORF1ab probe 2-1
gccctgacacacgttatgtgctcatgga

SEQ ID NO: 17 - DNA, 20 bp, synthetic construct
  source 1..20: mol_type = other DNA; note = an ORF1ab forward primer 2-2
atgaaagtttacgccctgac

SEQ ID NO: 18 - DNA, 23 bp, synthetic construct
  source 1..23: mol_type = other DNA; note = an ORF1ab reverse primer 2-2
ccgtgcctacagtactcagaatc

SEQ ID NO: 19 - DNA, 31 bp, synthetic construct
  source 1..31: mol_type = other DNA; note = an ORF1ab probe 2-2
atgtgctcatggatggctctattattcaatt

SEQ ID NO: 20 - DNA, 21 bp, synthetic construct
  source 1..21: mol_type = other DNA; note = an internal standard upstream primer
gaggtgggacttcagcatggc

SEQ ID NO: 21 - DNA, 23 bp, synthetic construct
  source 1..23: mol_type = other DNA; note = an internal standard downstream primer
tgtttcttttccttaaagtcaac

SEQ ID NO: 22 - DNA, 27 bp, synthetic construct
  source 1..27: mol_type = other DNA; note = an internal standard probe
agatttggacctgcgagcgggttctga

SEQ ID NO: 23 - DNA, 22 bp, synthetic construct
  source 1..22: mol_type = other DNA; note = China CDC SARS-CoV-2 N upstream primer
ggggaacttctcctgctagaat

SEQ ID NO: 24 - DNA, 22 bp, synthetic construct
  source 1..22: mol_type = other DNA; note = China CDCSARS-CoV-2 N downstream primer
cagacattttgctctcaagctg

SEQ ID NO: 25 - DNA, 20 bp, synthetic construct
  source 1..20: mol_type = other DNA; note = China CDCSARS-CoV-2 N probe
ttgctgctgcttgacagatt

SEQ ID NO: 26 - DNA, 20 bp, synthetic construct
  source 1..20: mol_type = other DNA; note = US CDCSARS-CoV-2_N1 upstream primer
gaccccaaaatcagcgaaat

SEQ ID NO: 27 - DNA, 24 bp, synthetic construct
  source 1..24: mol_type = other DNA; note = US CDCSARS-CoV-2_N1 downstream primer
tctggttactgccagttgaatctg

SEQ ID NO: 28 - DNA, 24 bp, synthetic construct
  source 1..24: mol_type = other DNA; note = US CDCSARS-CoV-2_N1 probe
accccgcattacgtttggtggacc

SEQ ID NO: 29 - DNA, 20 bp, synthetic construct
  source 1..20: mol_type = other DNA; note = US CDCSARS-CoV-2_N2 upstream primer
ttacaaacattggccgcaaa

SEQ ID NO: 30 - DNA, 18 bp, synthetic construct
  source 1..18: mol_type = other DNA; note = US CDCSARS-CoV-2_N2 downstream primer
gcgcgacattccgaagaa

SEQ ID NO: 31 - DNA, 23 bp, synthetic construct
  source 1..23: mol_type = other DNA; note = US CDCSARS-CoV-2_N2 probe
acaatttgcccccagcgcttcag

SEQ ID NO: 32 - DNA, 22 bp, synthetic construct
  source 1..22: mol_type = other DNA; note = US CDCSARS-CoV-2_N3 upstream primer
gggagccttgaatacaccaaaa

SEQ ID NO: 33 - DNA, 21 bp, synthetic construct
  source 1..21: mol_type = other DNA; note = US CDCSARS-CoV-2_N3 downstream primer
tgtagcacgattgcagcattg

SEQ ID NO: 34 - DNA, 24 bp, synthetic construct
  source 1..24: mol_type = other DNA; note = US CDCSARS-CoV-2_N3 probe
aycacattggcacccgcaatcctg

SEQ ID NO: 35 - DNA, 22 bp, synthetic construct
  source 1..22: mol_type = other DNA; note = HKU-N upstream primer
taatcagacaaggaactgatta

SEQ ID NO: 36 - DNA, 19 bp, synthetic construct
  source 1..19: mol_type = other DNA; note = HKU-N downstream primer
cgaaggtgtgacttccatg

SEQ ID NO: 37 - DNA, 20 bp, synthetic construct
  source 1..20: mol_type = other DNA; note = HKU-N probe
gcaaattgtgcaatttgcgg

SEQ ID NO: 38 - DNA, 193 bp, unidentified
  source 1..193: mol_type = genomic DNA; note = an area on the ORF1ab gene
ataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttcttta

SEQ ID NO: 39 - DNA, 20 bp, synthetic construct
  source 1..20: mol_type = other DNA; note = an ORF1ab forward primer 3
atgtatctaaagttgcgtag

SEQ ID NO: 40 - DNA, 23 bp, synthetic construct
  source 1..23: mol_type = other DNA; note = an ORF1ab reverse primer 3
gttactgaagtcattgagagcct

SEQ ID NO: 41 - DNA, 29 bp, synthetic construct
  source 1..29: mol_type = other DNA; note = an ORF1ab probe 3
atgtgctattacctcttacgcaatataat

SEQ ID NO: 42 - DNA, 219 bp, unidentified
  source 1..219: mol_type = genomic DNA; note = area on the N gene
tgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgca

SEQ ID NO: 43 - DNA, 21 bp, synthetic construct
  source 1..21: mol_type = other DNA; note = an N gene forward primer 2
actacctaggaactgggccag

SEQ ID NO: 44 - DNA, 21 bp, synthetic construct
  source 1..21: mol_type = other DNA; note = an N gene reverse primer 2
agcaggattgcgggtgccaat

SEQ ID NO: 45 - DNA, 30 bp, synthetic construct
  source 1..30: mol_type = other DNA; note = an N gene probe 2
acttccctatggtgctaacaaagacggcat

SEQ ID NO: 46 - DNA, 22 bp, synthetic construct
  source 1..22: mol_type = other DNA; note = an N gene forward primer 3
atgtctggtaaaggccaacaac

SEQ ID NO: 47 - DNA, 21 bp, synthetic construct
  source 1..21: mol_type = other DNA; note = an N gene reverse primer 3
agttccttgtctgattagttc

SEQ ID NO: 48 - DNA, 29 bp, synthetic construct
  source 1..29: mol_type = other DNA; note = an N gene probe 3
actgtcactaagaaatctgctgctgaggc